(19) United States
(12) Patent Application Publication
(10) Pub. No.: US 2022/0395168 A1
(43) Pub. Date: Dec. 15, 2022

(54) PHYSICIAN-CONTROLLED HANDLE FOR MEDICAL DEVICES

(71) Applicant: Cook Medical Technologies LLC, Bloomington, IN (US)

(72) Inventors: Meredith Lee, Winston-Salem, NC (US); Michael J. Brecht, Salisbury, NC (US)

(73) Assignee: Cook Medical Technologies LLC, Bloomington, IN (US)

(21) Appl. No.: 17/835,744

(22) Filed: Jun. 8, 2022

Related U.S. Application Data
(60) Provisional application No. 63/209,804, filed on Jun. 11, 2021.

(51) Int. Cl. A61B 1/12; A61B 1/00

(52) U.S. Cl. CPC A61B 1/12; A61B 1/00066; A61B 1/00128; A61B 1/00137; A61B 2017/00296

(57) ABSTRACT

A medical device includes a handle that includes a handle body longitudinally extending from a proximal end to a distal end; a distal connector configured to connect to a proximal end of an elongate outer member; a lumen longitudinally extending through the handle body, the lumen configured to have an elongate inner member movably disposed therein, and to permit the elongate inner member to distally extend past the distal end to within the elongate outer member and to proximally extend past the proximal end; and an inner member controller configured to longitudinally move relative to the handle body to control movement of the elongate inner member relative to the elongate outer member. The handle may be a distal handle of the medical device, which further includes a proximal handle. Both handles may control movement of the elongate inner and outer members.

### CROSS-REFERENCE TO RELATED APPLICATIONS

This application claims the benefit of U.S. Provisional Application No. 63/209,804, filed Jun. 11, 2021. The contents of U.S. Provisional Application No. 63/209,804 are hereby incorporated by reference in their entirety.

### TECHNICAL FIELD

The present invention relates generally to medical devices, and more particularly to clips.

### BACKGROUND

Endoscopic medical devices often include an elongate tubular outer member and an elongate inner member movably disposed within the elongate tubular member. The distal end of the inner member includes a distal element (such as, a clip, forceps, or a snare) for performance of a surgical procedure within a patient. A handle is connected to proximal ends of the outer and inner members to control longitudinal and axial movement of one member relative to the other, as well as operation of the distal element.

To perform a surgical procedure, the outer and inner members are advanced through a working channel of an endoscope until a distal end of the medical device is at a target treatment site within a patient. Once the distal end at the treatment site, the distal element is manipulated with the handle to perform a desired action on tissue at the treatment site.

The surgical procedure is typically performed with two people. One person, usually a physician, holds the endoscope with one hand and longitudinally advances and retracts the inner and outer members through the working channel by grasping the outer member with his/her other hand. A second person, usually a nurse technician, holds and controls the handle in order to control the relative movement of the inner and outer members and operation of the distal element. The second person controls the handle in accordance with instructions given orally by the first person.

More and more, physicians desire to control the relative movement of the inner and outer members and the distal element at the treatment site, rather than issue instructions to the nurse technician. However, when the distal end is at the treatment site, a length of the inner and outer members extending from the proximal opening of the endoscope to the handle is usually too great, such that the physician cannot effectively operate the handle while also holding the endoscope. As such, ways to provide the physician with control of the outer and inner members while also being able to hold the endoscope may be desirable.

### BRIEF SUMMARY

The present description describes medical devices that include a handle that an operator can hold and control while holding an endoscope. The handle is configured to control relative longitudinal movement and/or axial rotational movement of elongate inner and outer members, including during times that the inner and outer members are extending within the endoscope to a treatment site within the patient. The present further describes medical devices that include a handle system including a proximal handle and a distal handle positioned distal the proximal handle. Both the proximal handle and the distal handle are configured to control relative longitudinal movement and/or axial rotational movement of elongate inner and outer members to which the proximal and distal handles are operably connected.

In one embodiment, a medical device includes: a handle that includes: a handle body longitudinally extending from a proximal end to a distal end; a distal connector integrated with the distal end, the distal connector configured to connect to a proximal end of an elongate outer member; a lumen longitudinally extending through the handle body, the lumen configured have an elongate inner member movably disposed therein, and to permit the elongate inner member to distally extend past the distal end to within the elongate outer member and to proximally extend past the proximal end; and an inner member controller longitudinally disposed about the handle body between the proximal end and the distal end, the inner member controller configured to control at least one movement of the elongate inner member relative to the elongate outer member.

In another embodiment, a medical device includes: an elongate outer member longitudinally extending from a proximal portion to a distal portion of the medical device; an elongate inner member longitudinally extending within the elongate outer member from the proximal portion to the distal portion; a proximal handle configured to control at least one movement of the elongate inner member relative to the elongate outer member; and a distal handle configured to control the at least one movement of the elongate inner member through the elongate outer member.

In another embodiment, a method includes: distally advancing an elongate outer member and an elongate inner member of a medical device through a proximal opening of a working channel of an endoscope; upon distally advancing the elongate outer and inner members, positioning a distal handle of the medical device closer to the proximal opening than a proximal handle of the medical device; and with the distal handle, moving the elongate inner member relative to the elongate outer member.

Other embodiments are possible, and each of the embodiments can be used alone or together in combination. Accordingly, various embodiments will now be described with reference to the attached drawings.

### BRIEF DESCRIPTION OF THE DRAWINGS

FIG. 1A shows a cross-sectional side view of an embodiment of a medical device including a handle connected to an elongate inner member extending through a handle body of the handle.

FIG. 1B shows a cross-sectional side view of the medical device of FIG. 1A, with an inner member controller of the handle distally moved to distally advance the inner member and a distal element relative to an elongate outer member.

FIG. 1C shows a cross-sectional side view of the medical device of FIG. 1A, with an inner member controller of the handle proximally moved to proximally retract the inner member and a distal element relative to an elongate outer member.

FIG. 2 shows a proximal view of an embodiment of a proximal portion of a medical device including a handle connected to an elongate inner member extending through a handle body of the handle.

FIG. 3 shows a cross-sectional side view of an embodiment of a medical device including a handle connected to an elongate inner member extending through a handle body of the handle, where the handle body axially rotates both the inner member and an elongate outer member.

FIG. 4 shows a perspective view of a medical system including an endoscope and a medical device having two handles including a proximal handle and a distal handle.

FIG. 5A shows a cross-sectional side view of an embodiment of a medical device including a handle configured to selectively engage with an inner member extending through the handle, with a radially inner end of an inner member controller in an engaged position.

FIG. 5B shows a cross-sectional side view of the medical device of FIG. 5B, with the radially inner end in a disengaged position.

### DETAILED DESCRIPTION

The present description describes various embodiments of medical devices and medical systems that include a handle, and related methods that include operating a handle, for controlling movement, including relative longitudinal movement and/or axial rotational movement, of elongate inner and outer members of a medical device. During an endoscopic medical procedure, the handle may be positioned relatively close to a proximal opening of a working channel of an endoscope after distal ends of the outer and inner members have reached a treatment site within a patient, such that an operator is able to handle both the endoscope and the handle.

In various embodiments, the handle is a secondary or distal handle of a handle system that also includes a primary or proximal handle connected to proximal ends of the outer and inner members. Both the primary handle and the secondary handle may be configured to control relative longitudinal movement of the outer and inner members. While the operator is handling and controlling the distal handle, a second operator may merely hold the proximal handle at a position that minimizes stress and tension on proximal ends of the outer and inner members, without inhibiting or preventing the distal handle from controlling movement of the outer and inner members. However, if desired, the proximal handle can take over control of the movement of the outer and inner members.

Though use of the handle of the present description, an operator handling the endoscope, which is often a physician, can simultaneously control relative longitudinal and/or rotational movement of the outer and inner members and a distal member of the medical device used to perform an operation on tissue at the treatment site within the patient. In turn, the control of the movement of the outer and inner members does not have to depend on communication between a first operator controlling the endoscope and combined longitudinal movement of the outer and inner members and a second operator controlling relative longitudinal and rotational movement of the outer and inner members. This, in turn, may allow for quicker and more precise and accurate placement, movement, and/or deployment of the distal member at the treatment site. Various example embodiments of the handle are now described.

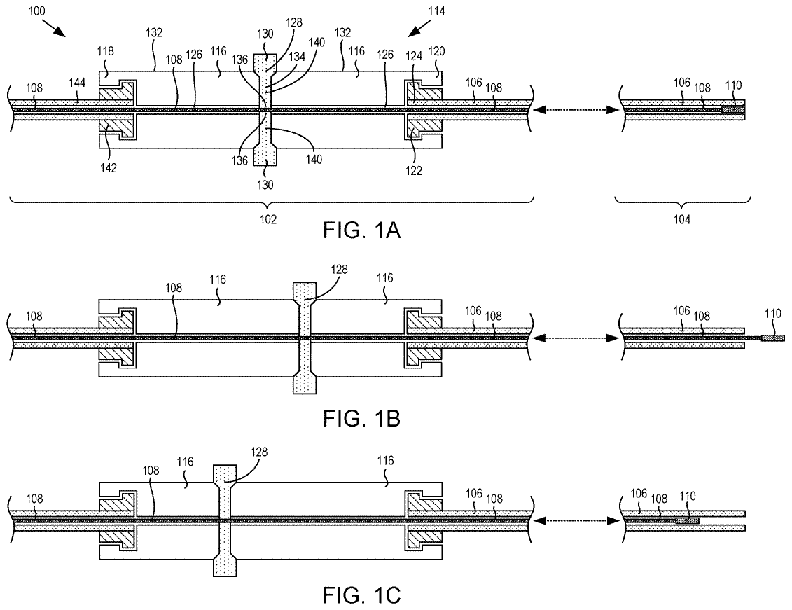

FIG. 1A shows a cross-sectional side view of an example embodiment of a medical device 100 extending from a proximal portion 102 to a distal portion 104. The medical device 100 includes an elongate outer member 106 and an elongate inner member 108, each extending from the proximal portion 102 to the distal portion 104.

The elongate inner member 108 is movably disposed within (e.g., within a lumen of) the elongate outer member 106. The outer and inner members 106, 108 may be configured to longitudinally move relative to each other. Such relative longitudinal movement used or leveraged to perform a medical procedure. For example, in the embodiment shown in FIG. 1A, the medical device 100 may further include a distal element 110 that is configured to perform an action on tissue at a treatment site within a patient associated with the medical procedure. Non-limiting examples of the distal element include a clip or clip assembly (e.g., a hemostatic clip or clip assembly used to achieve hemostasis at a bleeding site), forceps, an electrode or electrode assembly, a snare, a distal end of a needle knife, or a cutting edge (e.g., of a sphincterotome). The distal element 110 may be attached or connected to the inner member 108, such that longitudinal and rotational movement of the inner member 108 correspondingly controls longitudinal and rotational movement of the distal element 110. Accordingly, relative longitudinal movement of the outer and inner members 106, 108 may be used to extend the distal element 110 distally past a distal end 112 of the outer member 108 in order to expose the distal element 110 to the tissue at the treatment, and to proximally retract the distal element 110 to within the outer member 108.

The medical device 100 may further include a handle 114 at the proximal portion 102 that is configured to control relative longitudinal movement of the outer and inner members 106, 108. The handle 114 may include a handle body 116 that longitudinally extends from a proximal end 118 to a distal end 120. The handle 114 may further include a distal connector 122 integrated with the distal end 120 of the handle body 116. The distal connector 122 is configured to connect to, such as by gripping, grasping or otherwise being attached to, a proximal end 124 of the outer member 106.

In addition, the handle 114 includes a lumen 126 longitudinally extending through the handle body 116 from the proximal end 118 to the distal end 120. The lumen 126 is configured, such as being sized, to have the inner member 108 movably disposed therein. In addition, the lumen 126 is configured to permit the inner member 108 to distally extend past the distal end 120 of the handle body 116 to within the outer member 108, and to proximally extend past the proximal end 118 of the handle body 116.

Also, the handle 114 includes an inner member controller 128 longitudinally disposed about and integrated with the handle body 116 between the proximal end 118 and the distal end 120. The inner member controller 128 is configured to longitudinally move relative to the handle body 116 in order to control longitudinal movement of the inner member 108 relative to the outer member 106. In particular example embodiments, such as the one shown in FIG. 1, proximal movement of the inner member controller 128 relative to the handle body 116 proximally moves the inner member 108 relative to the outer member 106, and distal movement of the inner member controller 128 relative to the handle body 116 distally moves the inner member 108 relative to the outer member 106. Correspondingly, for embodiments where the medical device 100 includes a distal element 110, longitudinal movement of the inner member controller 128 longitudinally moves the distal element 110 in order to control the operation of the distal element 110 for performance of a medical operation on a patient. FIG. 1B shows the inner member controller 128 moved distally from its position in FIG. 1A relative to the handle body 116, which correspondingly distally moved the inner member 108 and the distal element 110 relative to the outer member 106. FIG. 1C shows the inner member controller 128 moved proximally from its positions in FIG. 1A and 1B relative to the handle body 116, which corresponding proximally moved the inner member 108 and the distal element 110 relative to the outer member 106.

Additionally, in particular embodiments such as shown FIG. 1A, the inner member controller 128 includes a radially outer portion 130 that radially protrudes from, or extends beyond, an outer surface 132 of the handle body 116. The radially outer portion 130 is a part of the inner member controller 128 that an operator of the handle 114 can contact or grasp with his/her hand (e.g., his/her thumb) to longitudinally move the inner member controller 128 relative to the handle body 116. Upon contacting or grasping the radially outer portion 130, the operator can longitudinally move or slide the radially outer portion 130 over the outer surface 132 of the handle body 116.

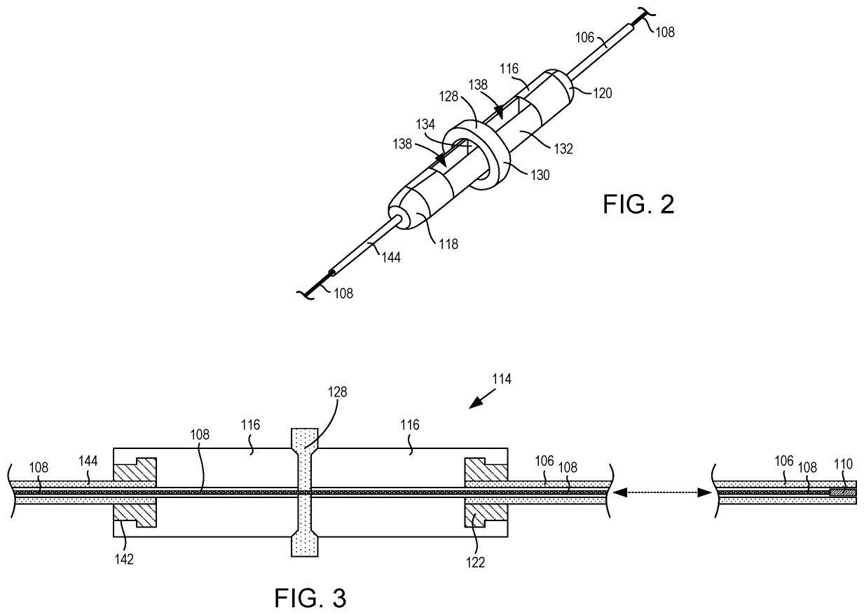

FIG. 2 shows a perspective view of the handle 102. For at least some embodiments of the handle 102, such as the one shown in FIG. 2, the handle body 116 is a substantially cylindrical structure and/or has a substantially circular axially cross-section about a longitudinal axis of the handle 102, at least over a portion of the handle body 116 over which the inner member controller 128 longitudinally moves. Additionally, the radially outer portion 130 of the inner member controller 128 is a substantially annular structure concentric with the cylindrical handle body 116. In combination, the cylindrical handle body 116 and the annular radially outer portion 130 provide the handle 102 with rotational symmetry, such that an operator can grasp and operate the handle 102 the same way for any rotational position of the handle 102 about its longitudinal axis. In other words, an operator does not have to grasp the handle 102 according to any one particular rotational position in order to optimally grasp and operate the handle 102. Other embodiments may not have such rotational symmetry or have at least partial rotational symmetry. For example, in other embodiments, the handle body 116 and/or the radially-outer outer portion 130 may have polygonal (e.g., rectangular, hexagonal, triangular, etc.) or elliptical axial cross-sections, as non-limiting examples.

With reference also to FIG. 1A, the inner member controller 128 may also include a radially inner portion 134 that radially extends inward from the radially outer portion 130 to the inner member 108. A radially inner end 136 of the radially inner portion 134 contacts the inner member 108 to allow the inner member controller 128 to control longitudinal movement of the inner member 108. In some embodiments, such as shown FIG. 1A, the inner member controller 128 is fixedly engaged with, or attached to, the inner member 108, in that the radially inner end 136 is fixedly engaged with or attached to the inner member 108.

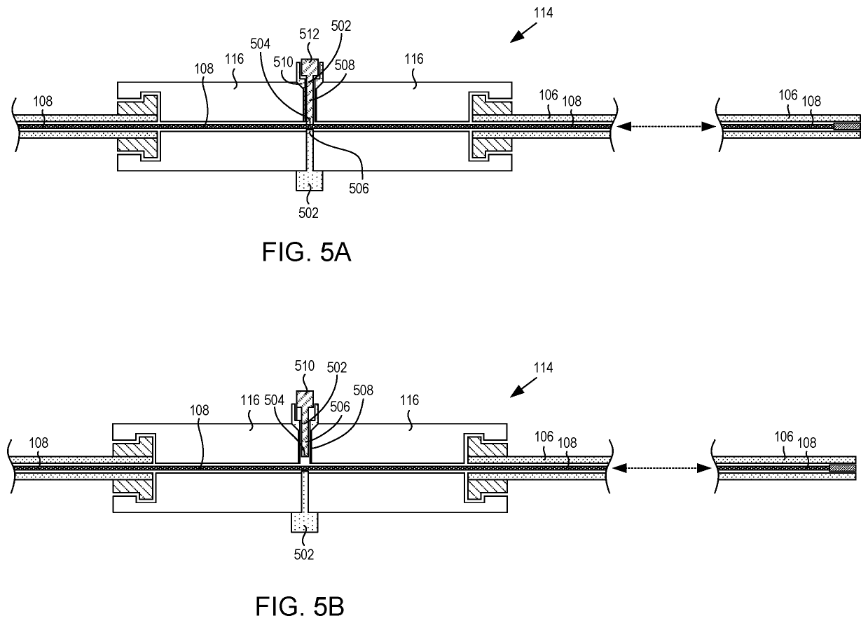

In other embodiments, such as shown in FIGS. 5A and 5B, instead of having an inner member controller 128 fixedly engaged or in contact with the inner member 108, the handle 114 includes an inner member controller 502 that selectively engages with the inner member 108. In this way, the inner member controller 502 provides the handle 114 with the ability to selectively control movement of the inner member 108.

In further detail, the inner member controller 502 in FIGS. 5A and 5B includes a radially inner end that includes two portions, including a first radially inner end portion 504 and a second radially inner end portion 506. The first radially inner end portion 504 is configured to selectively engage with, or attach to, the inner member 108. As shown in FIGS. 5A and 5B, the first radially inner end portion 504 may be part of, or at least operatively coupled to, an actuator (or actuator rod) 508 that is disposed and radially movable within an outer housing 510 of the inner member controller 502. The actuator 508 and radially inner end portion 504 may be movable between an engaged position and a disengaged position. FIG. 5A shows the actuator 506 and radially inner end portion 504 in the engaged position, and FIG. 5B shows the actuator 506 in the disengaged position.

Referring particularly to FIG. 5A, in the engaged position, the first radially inner end portion 504 is engaged or in contact with the inner member 108, such that longitudinal movement of the inner member controller 502 relative to the handle body 116 controls longitudinal movement of the inner member 108 relative to the outer member 106 and axial rotational movement of the inner member controller 502 and/or the handle body 116 controls axial rotational movement of the inner member 108. Referring particularly to FIG. 5B, in the disengaged position, the first radially inner end portion 504 is disengaged or not in contact with the inner member 108, such that longitudinal movement of the inner member controller 502 relative to the handle body 116 is unable to control longitudinal movement of the inner member 108 relative to the outer member 106, and axial rotational movement of the inner member controller 502 and/or the handle body 116 is unable to control axial rotational movement of the inner member 108.

Various ways of configuring the inner member controller 502 with the actuator 508 may be possible. For example, a radially outer end 512 of the actuator 508 may be configured as a button integrated with a spring-and-lock mechanism, similar to how a pen uses a button and spring-and-lock mechanism to withdraw and expose the writing tip into and out of the pen housing. Accordingly, when the actuator 508 is in the engaged position, the actuator 508 may be locked in the engaged position by the spring-and-lock mechanism. To move the actuator 508 into the disengaged position, an operator may press “down” (i.e., radially inward) on the radially outer end 512, which causes the spring-and-lock mechanism to unlock the actuator 508 in the engaged position, which in turn allows the spring to radially move the actuator 508 away from the inner member 108 into the disengaged position. In addition, when the actuator 508 is in the disengaged position, to move the actuator 508 into the engaged position, an operator may similarly press “down” on the radially outer end 512, which causes the spring-and-lock mechanism to lock the actuator 508 in the engaged position.

The second radially inner portion 506 may be fixedly disengaged from the inner member 108 so as not to interfere with the selective engagement of the first radially inner portion 504. However, the second radially inner portion 506 may be sufficiently close to the inner member 108 so as to provide a counter force to the “downward” or radially inward force that the first radially inner portion 504 exerts on the inner member 108 to facilitate the ability of the actuator 508, in conjunction with the spring-and-lock mechanism, to operate in the engaged and disengaged positions.

Referring to FIG. 2, the handle body 116 may include a recess or channel 138 that provides or functions as a track in which the radially inner portion 134 is disposed and through which the radially inward portion 134 longitudinally moves between the proximal and distal ends 118, 120 of the handle body 116. Additionally, the channel 138 radially extends from the outer surface 132 to an axially central area of the handle body 116 to allow the radially inner portion 134 to connect to, such as by contacting and/or fixedly attaching to, the inner member 108.

For at least some embodiments, such as shown in FIG. 1A, the radially inner portion 134 may include a plurality of radially inner portion elements 140 circumferentially spaced apart from each other about the longitudinal axis of the handle 102. Each radially inner portion element 140 may radially extend inward to connect or attach to the inner member 108. For such embodiments, the handle body 116 may include a plurality of channels 138, each configured to have one of the radially inner portion elements 140 movably disposed therein.

Additionally, for at least some embodiments as shown in FIG. 1A, the handle 114 may further include a proximal connector 142 configured to connect to, such as by gripping, grasping or otherwise being attached to, a second elongate outer member 144 of the medical device 100. Similar to the distal connector 122, the proximal connector 142 may be integrated with the proximal end 118 such that the inner member 108 proximally extends to within (e.g., within a lumen of) the second elongate outer member 144 as it proximally extends past the proximal end 118 of the handle body 116. For such embodiments, the outer member 106 may be considered a first or distal outer member portion, and the second outer member 144 may be considered a proximal outer member portion, of an overall elongate outer member of the medical device 100. The handle 114 effectively separates or divides the overall elongate outer member into its two outer member portions 106, 144, such that the proximal end 124 of the distal outer member portion 106 connects to the distal end 120 of the handle body 116, a distal end of the proximal outer member portion 144 connects to the proximal end 118 of the handle body 116, and the inner member 108 longitudinally extends through each of the proximal outer member portion 144, the handle 102, and the distal outer member portion 106.

In addition, during operation, an operator may desire to axially rotate the inner member 108 and/or the distal element 110, clockwise or counter-clockwise, about the longitudinal axis. To do so, the operator may axially rotate the handle 114, including the handle body 116 and/or the inner member controller 128, in the desired direction, which axially rotates the inner member 108 and the distal element 110. The handle body 116 and the inner member controller 128 may be configured such that they cannot axially rotate independent of one another. Accordingly, axial rotational movement of the handle body 116 axially rotates the inner member controller 128, and vice versa.

In some embodiments, such as shown in FIG. 1A, axial rotation of the handle 114, including the handle body 116 and/or the inner member controller 128, axially rotates the inner member 108 without rotating the outer member 106. In other words, axial rotation of the handle body 116 and/or the inner member controller 128 axially rotates the inner member 108 independent of, and/or relative to, the outer member 106. For such embodiments, the distal connector 122 is integrated with the handle body 116 such that distal connector 122 cannot longitudinally or axially separate from each other, but the handle body 116 and the distal connector 122 are configured to axially rotate relative to each other. For example, the distal connector 122 is positioned within the handle body 116 such that it can axially rotate within the handle body 116. Any of various configurations to provide relative axial rotation between the handle body 116 and the distal connector 122 may be possible. For example, as shown in FIG. 1A, a proximal end of the distal connector 122 may include a flange that is configured to abut an inner shoulder at the distal end 120 of the handle body 116, which prevents the distal connector 122 from axially separating from the handle body 116. Otherwise, the handle body 116 and the distal connector 122 are not physically connected to each other such that axial rotation of the handle body 116 and/or the inner member controller 128 does not cause axial rotation of the distal connector 122, and in turn the outer member 106. For embodiments that include a proximal connector 142 connected to a proximal outer member 144, such as in FIG. 1A, the proximal connector 142 may be similarly integrated with the handle body 116 like the distal connector 122, such that axial rotation of the handle body 116 and/or the inner member controller 128 does not axially rotate the proximal outer member 144, and in turn, the inner member 108 axially rotates independent of, and/or relative to the proximal outer member 144.

For other example embodiments, such as shown in FIG. 3, axial rotation of the handle 114, including the handle body 116 and/or the inner member controller 128, axially rotates both the outer member 106 and the inner member 108. For such embodiments, the distal connector 122 may be fixedly attached to the handle body 116, such that distal connector 122 and the handle body 116 are configured to not axially rotate relative to each other. In turn, axial rotation of the handle body 116 axially rotates the distal connector 122, and in turn axially rotates the outer member 106 along with the inner member 108. For such of these embodiments where the handle 114 also includes a proximal connector 142 connected to a proximal outer member 144, the proximal connector 142 may be similarly fixedly attached to the handle body, such that axial rotation of the handle body 116 axially rotates the proximal connector 142, and in turn axially rotates the proximal outer member 144 along with the inner member 108.

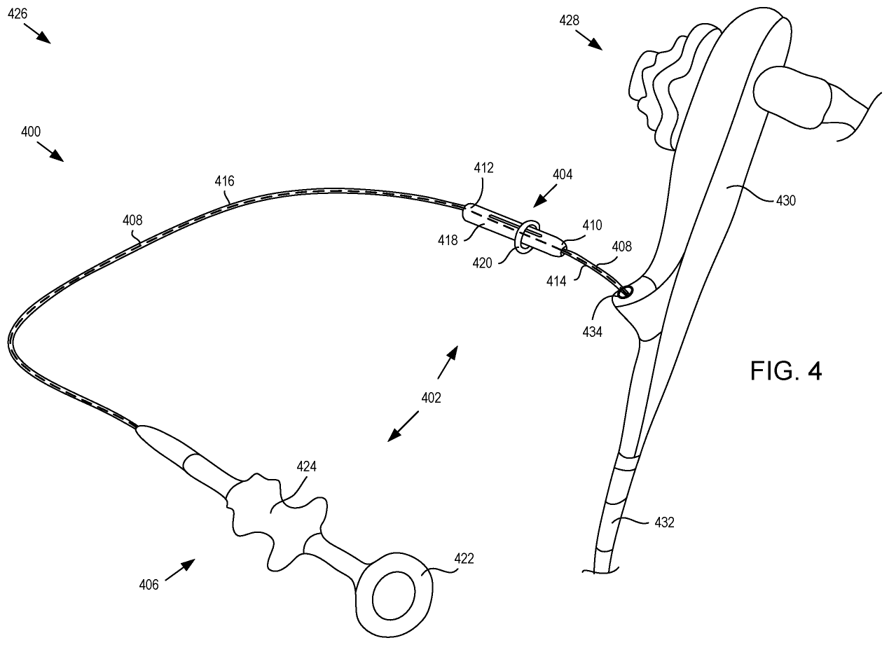

FIG. 4 shows an example embodiment of a medical device 400 that includes a handle system 402 including a handle 404, which may be configured according to any of, including having any of the features of, the embodiments of the handle 114 shown and described with reference to FIGS. 1A-3. In the handle system 402 of FIG. 4, the handle 404 is one of a plurality or a pair of handles, and may be referred to as a secondary or a distal handle 404 of the handle system 402. As shown in FIG. 4, the plurality of handles of the handle system 402 further includes a primary or a proximal handle 406 positioned proximal the distal handle 404.

Similar to the embodiments of FIGS. 1A-3, the medical device 400 includes an elongate inner member 408 that longitudinally extends through the distal handle 404, extending distally past a distal end 410 of the distal handle 404, and proximally past a proximal end 412 of the distal handle 404. Although not shown in FIG. 4, the inner member 408 may distally extend to a distal portion that includes a distal element configured to perform an action on tissue at a treatment site, like the inner member 108 distally extending to a distal element 110 in the embodiments of FIGS. 1A-3. In addition, similar to the elongate outer member 106 in the embodiments of the medical device 100 in FIGS. 1A-3, the medical device 400 of FIG. 4 may include an elongate distal outer member 414 longitudinally extending from, and connected to, the distal end 410 of the handle 404. The portion of the inner member 408 extending distally past the distal end 410 is movably disposed within the distal outer member 414. Though not shown in FIG. 4, the distal outer member 414 may distally extend with the inner member 408 to a distal portion, which can be moved to a treatment site within a patient.

Also, similar to the elongate proximal outer member 144 in the embodiments of FIGS. 1A-3, the medical device 400 of FIG. 4 may include an elongate proximal outer member 416 longitudinally extending from, and connected to, the proximal end 412. The portion of the inner member 408 extending proximally past the proximal end 412 is movably disposed within the proximal outer member 416. Like the embodiments of the handle 114 in FIGS. 1A-3, the distal handle 404 includes a handle body 418 and an inner member controller 420 configured to longitudinally move relative to the handle body 418 to control longitudinal movement of the inner member 408 relative to the distal outer member 408 and the proximal outer member 416.

In addition, as shown in FIG. 4, each of the inner member 408 and the proximal outer member 416 may proximally extend to, and terminate with, the proximal handle 406, such that proximal ends of each of the inner member 408 and the proximal outer member 416 are connected to the proximal handle 406. Like the distal handle 404, the proximal handle 406 is configured to control relative longitudinal movement of the inner member 408 relative to the distal and proximal outer members 414, 416. In the example embodiment in FIG. 4, the proximal handle 406 includes a thumb ring 422 and a finger-gripping portion 424. The thumb ring 422 and the finger-gripping portion 424 are configured to longitudinally move relative to each other. An operator grasps the proximal handle 406 by inserting his/her thumb through the thumb ring 422, and positioning at least two of his/her fingers over the finger-gripping portion 424. Upon doing so, the operator can longitudinally move the thumb ring 422 and the finger-gripping portion 424 relative to each other by moving his/her thumb and fingers closer and farther away from each other. The thumb ring 422 is operably connected to the inner member 408, and the finger-gripping portion 424 is operably connected to the proximal outer member 416. In turn, relative longitudinal movement between the thumb ring 422 and the finger-gripping portion 424 longitudinally moves the inner member 408 relative to the proximal outer member 416, the distal handle 404, and the distal outer member 414. The configuration of the proximal handle 406 shown in FIG. 4 is merely an example,

Accordingly, both the distal handle 404 and the proximal handle 406 are configured to control relative longitudinal movement of the inner member 408 relative to the distal and proximal outer members 414, 416. Generally, the distal and proximal handles 404, 406 are handled by different operators during operation. For example, a first operator, such as a physician, may handle the distal handle 404, and a second operator, such as a nurse technician, may handle the proximal handle 406. Desirably, the first operator and the second operator alternatingly have control of the distal and proximal handles 404, 406, respectively. That is, when the first operator is controlling or operating the distal handle 404, the second operator is not controlling or operating the proximal handle 406. Instead, the second operator may not be handling the proximal handle 406 at all, or may handle the proximal handle 406 in a way that does not exert relative longitudinal forces on the thumb ring 422 and the finger-gripping portion 424 so as to inhibit or restrict the ability of the distal handle 404 to control relative longitudinal movement of the inner and outer members 408, 414, 416. Similarly, when the second operator is controlling or operating the proximal handle 406, the first operator is not controlling or operating the distal handle 404. Instead, the first operator may not be handling the distal handle 404 at all, or may handle the distal handle 404 in a way that does not exert relative longitudinal forces on the handle body 418 and the inner member controller 420 so as to inhibit or restrict the ability of the proximal handle 406 to control relative longitudinal movement of the inner and outer members 408, 414, 416.

A method of operation of the handle system 402 is now described in further detail in conjunction with performance of a medical procedure or operation, such as an endoscopic medical procedure, that uses a medical system 426 that includes the medical device 400 and an endoscope 428. In general, the endoscope 428 is used, among other things, to guide or facilitate the movement of a distal portion of the medical device 400 to a treatment site within a patient that is the subject of the medical operation. The endoscope 428 includes a handle 430 and an insertion tube 432 that is inserted into a patient. The insertion tube 432 extends from a proximal end connected to the handle 430 to a distal end (not shown). The insertion tube 432 has a longitudinal length sufficient to reach the treatment site within the patient. The endoscope 426 further includes a working channel longitudinally extending through the insertion tube 432 and that proximally extends to a proximal opening 434 at a portion of the handle 430.

An operator, such as a physician, may insert the insertion tube 432 into an opening of a patient and distally advance the insertion tube 436 within the patient until a distal end of the insertion tube 436 reaches a desired location at or near the treatment site. The operator may then insert a distal end of the medical device 400, which may include distal ends of the inner member 408 and distal outer member 414, into the working channel proximal opening 434, and distally advance the distal outer member 414 and a portion of the inner member 408 distal the distal handle 404 through the working channel until the distal end of the medical device 400 reaches the treatment site. The operator may do so by grasping and distally pushing the distal outer member 414 through the working channel, and the portion of the inner member 408 distal the distal handle 404 may move with the distal outer member 414. While the operator is distally advancing the distal outer member 414 with one hand, the operator may be simultaneously holding the endoscope handle 430 with his/her other hand.

Upon distally advancing the distal outer member 414 and the portion of the inner member 408 distal the distal handle 404, the distal handle 404 may be positioned closer to the proximal opening 434 than the proximal handle 406, as measured over a longitudinal length of the medical device 400, as shown in FIG. 4. Accordingly, the distal outer member 414 may have a longitudinal length that is sufficiently long such that its distal end can reach the treatment site within the patient while a proximal portion of the distal outer member 414 remains outside of the endoscope 428, as shown in FIG. 4. In addition, the length of the distal outer member 414 may be sufficiently short such that when its distal end is at the treatment site, the distal end 410 of the distal handle 404 is sufficiently close to the working channel proximal opening 434 (e.g., within an average arm span of an adult person) such that the operator is able to simultaneously grasp and handle both the endoscope handle 430 and the distal handle 404, without the proximal portion of the distal outer member 414 that is outside of the endoscope 428 being substantially bent (such as in a U-shape) or coiled in one or more turns, such as shown in FIG. 4. This allows the operator to simultaneously hold and operate the endoscope handle 430 with one hand, and hold and operate the distal handle 404 with his/her other hand.

Upon grasping the distal handle 404 with one hand, the operator controls the longitudinal and axial rotational movements of the inner member 408 and the distal outer member 414 as desired, via operation of the distal handle 404, in order to perform the medical operation at the treatment site, all while holding the endoscope 428 with the other hand. In particular, the operator controls the relative longitudinal movement between the inner member 408 and the distal outer member 414 by moving the inner member controller 420 relative to the handle body 418, as previously described. In addition or alternatively, the operator controls a combined longitudinal movement of the inner and distal outer members 408, 414 (the combined longitudinal movement being where the inner and distal outer members 408, 414 longitudinal move together as opposed to relative to one another) by moving all of the components of the distal handle 404 together (as opposed to moving the inner member controller 420 relative to the handle body 418). The distal handle 404 may control the combined longitudinal movement effectively because the relatively short length of the inner member 408 and the distal outer member 414, between the handle distal end 410 and the endoscope proximal opening 434, may prevent them from kinking as the distal handle 404 is moved. Additionally, while holding the endoscope handle 430, the operator may also control axial rotation of the inner member 408 relative to the distal outer member 414, or axial rotation of both the inner member 408 and the distal outer member 414, by axially rotating the distal handle 404, including the handle body 418 and the inner member controller 420.

In general, the distal handle 404 may provide an operator holding the endoscope 426, such as a physician, with complete control of the medical device 400 to successfully perform an intended operation or action at the treatment site. Doing so may eliminate the need for the operator holding the endoscope 428 to issue instructions to a second operator that is controlling the medical device 400 with the proximal handle 406. Providing the operator holding the endoscope 426 with complete control of the medical device 400 to perform the operation at the treatment site may allow for more accurate, precise, and quicker maneuvering of the distal end of the medical device 400 at the treatment site, and an overall improved medical procedure.

While the operator is controlling the distal handle 404, a second operator may hold the proximal handle 404 in a way that does not impede, or aims to minimize the degree it impedes on, the first operator's control of the distal handle 404. For example, the second operator may hold the proximal handle 404 without gripping the proximal handle 404 using the thumb ring 422 and the finger-gripping portion 424. Additionally, the second operator may hold or position the proximal handle 406 relative to the distal handle 404 to minimize stress on the inner member 408 and the proximal outer member 416, and/or minimize friction between the inner and proximal outer members 408, 416, so as not to impede the distal handle's 404 control of the relative longitudinal movement between the inner and distal outer members 408, 414. For example, the proximal handle 406 may be positioned at the same or substantially same height from a floor as the distal handle 404, in various embodiments of the method.

Additionally, in various embodiments of the method of operation, the first operator controlling the distal handle 404 may control the movements of the inner and outer members 408, 414, 416 for the entirety of the operation, while the second operator merely holds the proximal handle 406 at an optimum position relative to the distal handle 404. In other embodiments, the second operator controlling the proximal handle 406 may control the movements of the inner and outer members 408, 414, 416 for at least a portion of the operation. For example, the first operator handling and controlling the endoscope 428 may never grasp the distal handle 404, and all relative longitudinal movement and axial rotational movement of the inner and outer members 408, 414, 416 is performed using the proximal handle 406. In other embodiments, the first operator may control movement of the inner and outer members 408, 414, 416 using the distal handle 404 for a first portion of the operation, and the second operator may control movement of the inner and outer members 408, 414, 416 using the proximal handle 406 for a second portion of the operation. During the second portion, the first operator may provide commands, instructions, or guidance (such as audibly) to the second operator, and the second operator may control the proximal handle 406 according to the commands. For any of these embodiments, the operation may be defined over a continuous time period starting at a time that the distal end of the medical device 400 is inserted into the proximal opening 434 of the endoscope 428 and ending at a time that the distal end is withdrawn from the proximal opening 434 to outside the endoscope 428. Alternatively, the operation may be defined over a continuous time period starting at a time that the distal end of the medical device 400 arrives at the treatment site within the patient and ending at a time that the distal end is withdrawn from the treatment site. The inclusion of the two handles 404, 406 to control movement of the inner and outer members 408, 414, 416 provides increased flexibility to the degree of control, including more control, that the first operator holding the endoscope 428 has over the various actions performed during the medical operation, compared to other devices that include only a proximal handle at proximal ends of inner and outer members.

The subject matter of the present description may also relate, among others, to the following aspects:

A first aspect relates to a medical device comprising: a handle comprising: a handle body longitudinally extending from a proximal end to a distal end; a distal connector integrated with the distal end, the distal connector configured to connect to a proximal end of an elongate outer member; a lumen longitudinally extending through the handle body, the lumen configured to have an elongate inner member movably disposed therein, and to permit the elongate inner member to distally extend past the distal end to within the elongate outer member and to proximally extend past the proximal end; and an inner member controller longitudinally disposed about the handle body between the proximal end and the distal end, the inner member controller configured to control at least one movement of the elongate inner member relative to the elongate outer member.

A second aspect relates to the medical device of the first aspect, and further wherein the elongate outer member comprises a first elongate outer member, the handle further comprising a proximal connector integrated with the proximal end, the proximal connector configured to connect to a distal end of a second elongate outer member.

A third aspect relates to the medical device of any preceding aspect, and further wherein the inner member controller comprises an annular radially outer portion disposed about the handle body.

A fourth aspect relates to the medical device of any preceding aspect, and further wherein a radial outer portion of the inner member controller is concentric with a cylindrical portion of the handle body.

A fifth aspect relates to the medical device of any preceding aspect, and further wherein the handle body comprises a longitudinally extending channel, and the inner member controller comprises a radially inner portion movably disposed within the channel, the radially inner portion comprising a radially inner end configured to connect to the elongate inner member.

A sixth aspect relates to the medical device of any preceding aspect, and further wherein the inner member controller and the handle body are configured to not axially rotate relative to each other.

A seventh aspect relates to the medical device of any preceding aspect, and further wherein the distal connector and the handle body are configured to axially rotate relative to each other.

An eighth aspect relates to the medical device of any of the preceding first through sixth aspects, and further wherein the distal connector and the handle body are configured to not axially rotate relative to each other.

A ninth aspect relates to the medical device of any of the preceding aspects, and further wherein the inner member controller is fixedly engaged with the inner member.

A tenth aspect relates to the medical device of any of the preceding first through eighth aspects, and further wherein the inner member controller is selectively engaged with the inner member.

An eleventh aspect relates to the medical device of any of the preceding aspects, and further wherein the at least one movement comprises a longitudinal movement.

A twelfth aspect relates to the medical device of any of the preceding first through tenth aspects, and further wherein the at least one movement comprises an axial rotational movement.

A thirteenth aspects relates to the medical device of any of the preceding first through tenth aspects, and further wherein the at least one movement comprises both a longitudinal movement and an axial rotational movement.

A fourteenth aspect relates to a medical device comprising: an elongate outer member longitudinally extending from a proximal portion to a distal portion of the medical device; an elongate inner member longitudinally extending within the elongate outer member from the proximal portion to the distal portion; a proximal handle configured to control at least one movement of the elongate inner member relative to the elongate outer member; and a distal handle configured to control the at least one movement of the elongate inner member through the elongate outer member.

A fifteenth aspect relates to the medical device of the fourteenth aspect fourteen, and further wherein the elongate outer member comprises a distal elongate outer member, a proximal end of the distal elongate outer member connected to a distal end of the distal handle, the medical device further comprising: a proximal elongate outer member, a distal end of the proximal elongate outer member connected to a proximal end of the distal handle and a proximal end of the proximal elongate outer member connected to the proximal handle, wherein the elongate inner member further longitudinally extends within the proximal elongate outer member.

A sixteenth aspect relates to the medical device of any of the preceding fourteenth or fifteenth aspects, and further wherein the at least one movement comprises a longitudinal movement.

A seventeenth aspect relates to the medical device of any of the preceding fourteenth or fifteenth aspects, and further wherein the at least one movement comprises an axial rotational movement.

An eighteenth aspect relates to the medical device of any of the preceding fourteenth or fifteenth aspects, and further wherein the at least one movement comprises both a longitudinal movement and an axial rotational movement.

A nineteenth aspect relates to the medical device of any of the preceding fourteenth through eighteenth aspects, wherein the distal handle comprises: a handle body; a distal connector connected to the proximal end of the distal elongate outer member; and a proximal connector connected to the distal end of the proximal elongate outer member, the handle body configured to axially rotate relative to each of the distal connector and the proximal connector.

A twentieth aspect relates to the medical device of any of the preceding fourteenth through nineteenth aspects, and further wherein the distal handle comprises: a handle body longitudinally extending from the proximal end to the distal end of the distal handle; and an inner member controller disposed about the handle body, the inner member controller comprising a radially inner end engaged with the elongate inner member.

A twenty-first aspect relates to the medical device of the twentieth aspect, and further wherein the radially inner end is fixedly engaged with the elongate inner member.

A twenty-second aspect relates to the medical device of the twentieth aspect, and further wherein the radially inner end is selectively engaged with the elongate inner member.

A twenty-third aspect relates to the medical device of any of the preceding twentieth through twenty-second aspects, and further wherein the inner member controller comprises an annular radially outer portion disposed about the handle body.

A twenty-fourth aspect relates to the medical device of the preceding twenty-third aspect, and further wherein the handle body comprises a cylindrical structure, the annular radially outer portion movably disposed over the cylindrical structure.

A twenty-fifth aspect relates to the medical device of any of the preceding fourteenth through twenty-fourth aspects, and further wherein the distal handle comprises: a handle body; and a lumen extending through the handle body, the elongate inner member movably disposed within the lumen and extending distally past a distal end of the handle body and proximally past a proximal end of the handle body.

A twenty-sixth aspect relates to a method comprising: distally advancing an elongate outer member and an elongate inner member of a medical device through a proximal opening of a working channel of an endoscope; upon distally advancing the elongate outer and inner members, positioning a distal handle of the medical device closer to the proximal opening than a proximal handle of the medical device; and with the distal handle, moving the elongate inner member relative to the elongate outer member.

A twenty-seventh aspect relates to the preceding twenty-sixth aspect, and further wherein moving the elongate inner member relative to the elongate outer member comprises longitudinally moving the elongate inner member relative to the elongate outer member.

A twenty-eighth aspect relates to the preceding twenty-seventh aspect, and further wherein longitudinally moving the elongate inner member relative to the elongate outer member comprises longitudinally moving an inner member controller relative to a handle body of the distal handle.

A twenty-ninth aspect relates to the method of the preceding twenty-seventh aspect, and further wherein the elongate outer member comprises a distal elongate outer member, the method further comprising: with the distal handle, longitudinally moving the elongate inner member relative to a proximal elongate outer member, the elongate inner member movably disposed within the proximal elongate outer member between the proximal handle and the distal handle.

A thirtieth aspect relates to the method of the preceding twenty-sixth aspect, and further wherein moving the elongate inner member relative to the elongate outer member comprises axially rotating the elongate inner member relative to the elongate outer member.

A thirty-first aspect relates to the method of the preceding thirtieth aspect, and further wherein axially rotating the elongate inner member relative to the elongate outer member comprises axially rotating the distal handle relative to the elongate outer member.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A medical device comprising:
a handle comprising:
a handle body longitudinally extending from a proximal end to a distal end;
a distal connector integrated with the distal end, the distal connector configured to connect to a proximal end of an elongate outer member;
a lumen longitudinally extending through the handle body, the lumen configured to have an elongate inner member movably disposed therein, and to permit the elongate inner member to distally extend past the distal end to within the elongate outer member and to proximally extend past the proximal end; and
an inner member controller longitudinally disposed about the handle body between the proximal end and the distal end, the inner member controller configured to control at least one movement of the elongate inner member relative to the elongate outer member.

2. The medical device of claim 1, wherein the elongate outer member comprises a first elongate outer member, the handle further comprising a proximal connector integrated with the proximal end, the proximal connector configured to connect to a distal end of a second elongate outer member.

3. The medical device of claim 1, wherein the inner member controller comprises an annular radially outer portion disposed about the handle body.

4. The medical device of claim 1, wherein a radial outer portion of the inner member controller is concentric with a cylindrical portion of the handle body.

5. The medical device of claim 1, wherein the handle body comprises a longitudinally extending channel, and the inner member controller comprises a radially inner portion movably disposed within the channel, the radially inner portion comprising a radially inner end configured to connect to the elongate inner member.

6. The medical device of claim 1, wherein the inner member controller and the handle body are configured to not axially rotate relative to each other.

7. The medical device of claim 1, wherein the distal connector and the handle body are configured to axially rotate relative to each other.

8. The medical device of claim 1, wherein the distal connector and the handle body are configured to not axially rotate relative to each other.

9. The medical device of claim 1, wherein the inner member controller is fixedly engaged with the inner member.

10. The medical device of claim 1, wherein the inner member controller is selectively engaged with the inner member.

11. The medical device of claim 1, wherein the at least one movement comprises a longitudinal movement.

12. The medical device of claim 1, wherein the at least one movement comprises an axial rotational movement.

13. The medical device of claim 1, wherein the at least one movement comprises both a longitudinal movement and an axial rotational movement.

14. A medical device comprising:
an elongate outer member longitudinally extending from a proximal portion to a distal portion of the medical device;
an elongate inner member longitudinally extending within the elongate outer member from the proximal portion to the distal portion;
a proximal handle configured to control at least one movement of the elongate inner member relative to the elongate outer member; and
a distal handle configured to control the at least one movement of the elongate inner member through the elongate outer member.

15. The medical device of claim 14, wherein the elongate outer member comprises a distal elongate outer member, a proximal end of the distal elongate outer member connected to a distal end of the distal handle, the medical device further comprising:
a proximal elongate outer member, a distal end of the proximal elongate outer member connected to a proximal end of the distal handle and a proximal end of the proximal elongate outer member connected to the proximal handle,
wherein the elongate inner member further longitudinally extends within the proximal elongate outer member.

16. The medical device of claim 14, wherein the at least one movement comprises a longitudinal movement.

17. The medical device of claim 14, wherein the at least one movement comprises an axial rotational movement.

18. The medical device of claim 14, wherein the at least one movement comprises both a longitudinal movement and an axial rotational movement.

19. The medical device of claim 14, wherein the distal handle comprises:
a handle body;
a distal connector connected to the proximal end of the distal elongate outer member; and
a proximal connector connected to the distal end of the proximal elongate outer member,
the handle body configured to axially rotate relative to each of the distal connector and the proximal connector.

20. The medical device of claim 14, wherein the distal handle comprises:
a handle body longitudinally extending from the proximal end to the distal end of the distal handle; and
an inner member controller disposed about the handle body, the inner member controller comprising a radially inner end engaged with the elongate inner member.

21. The medical device of claim 20, wherein the radially inner end is fixedly engaged with the elongate inner member.

22. The medical device of claim 20, wherein the radially inner end is selectively engaged with the elongate inner member.

23. The medical device of claim 20, wherein the inner member controller comprises an annular radially outer portion disposed about the handle body.

24. The medical device of claim 23, wherein the handle body comprises a cylindrical structure, the annular radially outer portion movably disposed over the cylindrical structure.

25. The medical device of claim 14, wherein the distal handle comprises:
a handle body; and
a lumen extending through the handle body, the elongate inner member movably disposed within the lumen and extending distally past a distal end of the handle body and proximally past a proximal end of the handle body.

26. A method comprising:
distally advancing an elongate outer member and an elongate inner member of a medical device through a proximal opening of a working channel of an endoscope;
upon distally advancing the elongate outer and inner members, positioning a distal handle of the medical device closer to the proximal opening than a proximal handle of the medical device; and
with the distal handle, moving the elongate inner member relative to the elongate outer member.

27. The method of claim 26, wherein moving the elongate inner member relative to the elongate outer member comprises longitudinally moving the elongate inner member relative to the elongate outer member.

28. The method of claim 27, wherein longitudinally moving the elongate inner member relative to the elongate outer member comprises longitudinally moving an inner member controller relative to a handle body of the distal handle.

29. The method of claim 27, wherein the elongate outer member comprises a distal elongate outer member, the method further comprising:
with the distal handle, longitudinally moving the elongate inner member relative to a proximal elongate outer member, the elongate inner member movably disposed within the proximal elongate outer member between the proximal handle and the distal handle.

30. The method of claim 26, wherein moving the elongate inner member relative to the elongate outer member comprises axially rotating the elongate inner member relative to the elongate outer member.

31. The method of claim 30, wherein axially rotating the elongate inner member relative to the elongate outer member comprises axially rotating the distal handle relative to the elongate outer member.
